(19) United States
(12) Patent Application Publication
(10) Pub. No.: US 2008/0147197 A1
(43) Pub. Date: Jun. 19, 2008

(54) BIODEGRADABLE OSTEOGENIC POROUS BIOMEDICAL IMPLANT WITH IMPERMEABLE MEMBRANE

(76) Inventor: William F McKay, Memphis, TN (US)

Correspondence Address: FOX ROTHSCHILD, LLP, 997 LENOX DRIVE, LAWRENCEVILLE, NJ, 08648, US

(21) Appl. No.: 11/610,957

(22) Filed: Dec. 14, 2006

(51) Int. Cl. A61F 2/28; B29C 39/10

(52) U.S. Cl. 623/23.51; 206/570; 264/279

(57) ABSTRACT

A biomedical implant is disclosed with osteogenic factors and a solid impermeable membrane occluding a portion of its surface for the generation of new bone growth at the target site of the implant. The implant is porous, bioresorbable, and forms a three dimensional architectural scaffold for the formation of new bone tissue. The implant is formed with a polymer or collagen, bone morphogenetic protein and ceramic particles.

### FIELD OF THE INVENTION

The present invention relates generally to the design of an implant depot with growth factors for the generation of new bone. Specifically, the invention relates to an implant used as a bone void filler and contains an effective composition and configuration for the surgical repair of bone void areas.

### BACKGROUND OF THE INVENTION

It is estimated there are more than 500,000 bone grafting procedures performed annually in the United States and these procedures approach a cost of $2.5 billion per year. Further, these numbers easily double on a world wide basis. Most of these bone graft procedures are performed with autograft or allograft tissues.

Autograft refers to bone tissue acquired from the same individual but from a location other than the intended target site. Usually the autograft bone tissue is harvested from the iliac crest, distal femur or proximal tibia. This harvested tissue is then placed on the injury site. This type of tissue is ideal since it possesses the necessary characteristics required for new bone growth without eliciting an immune reaction causing a rejection of the bone graft. The necessary characteristics for new bone growth are osteogenesis, osteoinduction and osteoconduction. However, harvesting autograft tissue requires additional surgeries and is often associated with donor site morbidity. The donor site complications can include inflammation, infection, and chronic pain. In some situations, the chronic pain may even outlast the pain associated with the original site of injury. Further, the supply of autografts may be limited in any particular individual.

Alternatives to autografts are the acquisition of bone tissue from other individuals or cadavers. With the use of allografts some of the shortcomings of autografts are eliminated. That is, the supply limitations is diminished along with a potential reduction in donor site morbidity or its elimination in the case a cadavers. However, there is still a certain risk of disease transmission, even with treated tissues, and an increased risk of an adverse immune response. Further, treated tissues to reduce the risk of disease transmission may affect the incorporation of the graft and its structural strength. That is, the treating process decreases the number of viable cells and proteins that influence the growth of new bone and further, the graft may not have the same structural strength in comparison to an autograft.

Both autografts and allografts have their drawbacks and therefore safer bone graft substitutes would be beneficial. These safer substitutes are usually constituted from non-bone derived materials. These safer substitutes ideally should be biocompatible, bioresorbable, osteoconductive, osteoinductive and osteogenic for the generation of new bone at the site of injury (i.e., at intended bone graft site). In addition, the implant should not be infiltrated by other surrounding soft tissue cells that may interfere with bone tissue growth. Ideally the implant should also provide an environment that is maximally conducive for new bone growth at the intended target site. Any soft tissue cells that infiltrate the porous implant surface will retard the process of new bone growth or even truncate the developmental pathway to new bone tissue. This type of problem may cause a severely weakened graft or even a non-union and hence a failed implant. Failed implants have increased morbidities, impose additional suffering upon patients, and increased costs for both patient and society.

### SUMMARY OF THE INVENTION

The instant invention uses a carrier matrix with bone-free derived materials, that is, ceramic particles. It also includes a natural or synthetic degradable material or polymer, preferably collagen, to form a porous resorbable sponge type of biomedical implant for the generation of new bone growth at the intended target site. It is an object of the present invention to provide a porous resorbable implant with a desirable three-dimensional architectural configuration fitted to the particular target site or bone void area. The three-dimensional architectural configuration provides a stable yet a sponge-like consistency to facilitate mechanical shaping and filling of the bone void areas. The porous implant is incorporated with an effective amount of a growth factor that preferably stimulates osteoblasts. The growth factor is preferably bone morphogenetic protein. The biomedical implant contains a particulate mineral having an average diameter of at least about 0.1 mm, but preferably about 1.0 mm, incorporated in collagen or a polymer and having a weight ratio of at least 3:1 relative to the collagen or polymer. More preferred compositions are even more highly mineralized and in some embodiments they constitute at least a weight ratio of 10:1.

U.S. patent application Ser. No. 09/923,116 (the '116 application), incorporated herein by reference in its entirety, describes an osteogenic implant including collagen and particulate minerals selected from a group consisting of bone particles and biocompatible synthetic calcium phosphate ceramics. The particular feature of the '116 application relates to the inclusion of osteogenic factors in a resorbable implant composition to fill bone voids. However, the '116 application did not address the issue of cellular fibrous tissue ingrowth into the implant, which interferes with new bone growth. This cellular tissue ingrowth slows the process of bone regeneration and also potentially weakens the graft and possibly causing a non-union. The present invention employs a solid impermeable membrane in addition to an osteogenic or growth factor and ceramic particles or materials. Osteogenic or osteogenesis refers to the general ability of the biomedical implant to generate new bone tissue whether with the interaction of host cells or imbedded stem cells in the biomedical implant. Osteogenesis or new bone growth may occur when the biomedical implant is imbedded with bone morphogenetic protein (BMP) and interacts with the host site.

Another object of the invention is to provide osteoinduction for new bone growth. That is, the porous biomedical implant, containing an effective amount of imbedded growth factors, recruits and transforms host cells for bone regeneration at the intended target site.

Still another object of the invention is to provide osteoconduction for new bone growth. The porous biomedical implant provides surfaces that are receptive for the growth of new bone tissue.

Yet another object of the invention is to provide selective tissue ingrowth to facilitate and enhance the proliferation of bone tissue cells for the growth of new bone. Preferably bone cells migrate to and proliferate in the porous implant with the least amount of surrounding soft tissue cell invasion of the biomedical implant. Selective tissue ingrowth may be achieved with an integral attachment of a solid relatively impermeable membrane to a surface portion of the biomedical implant.

Yet still another object of the invention is for the biomedical implant to provide guided tissue regeneration, particularly in many oral maxillofacial procedures, without the need for suturing or securing guided tissue regeneration sheets over the bone graft.

Yet still another object of the invention is to provide a degradable impermeable sheet or membrane pre-applied to the biomedical implant to produce a better seal then suturing separate sheets during the implant procedure and also to eliminate subsequent surgeries for the removal of the sheet or membrane.

### BRIEF DESCRIPTION OF THE DRAWINGS

FIG. 1 is a diagrammatic top view of the biomedical implant. The dotted line circumscribes the contours of the bone void which contains the biomedical implant. The solid line circumscribes the contour of the solid membrane.

FIG. 2 is a cross-sectional diagrammatical view of the implant indicating the bone void filler with a solid membrane attached at the surface.

FIG. 3A is a cross-sectional diagrammatical view of a bone void defect in alveolar bone.

FIG. 3B illustrates a cross-sectional diagrammatical view of the alveolar bone defect implant filling the bone void and a solid membrane sheet on the top surface of the implant.

FIG. 4A illustrates a cross-sectional diagrammatical view of the implant in a periodontal application applied to a bone defect area with the biomedical implant being pierced by a tooth structure.

FIG. 4B illustrates a cross-sectional diagrammatical view of a dental implant applied to the alveolar bone.

FIG. 5 illustrates a diagrammatic top view of another embodiment. The center circle represents a biological structure, such as a tooth, protruding through the implant. The surrounding top surface represents the solid membrane. The implant is below the solid membrane.

FIG. 6A illustrates a side view of a portion of a contoured alveolar bone with the application of the biomedical implant for restoration of part of the oral surface of the alveolar bone.

FIG. 6B illustrates a cross-sectional view through the center of the biomedical implant in FIG. 6A. FIG. 6B represents a defect situation in the alveolar bone filled with the biomedical implant.

FIG. 6C illustrates a cross-sectional view through the center of the biomedical implant in FIG. 6A. FIG. 6C represents a situation for the restoration of the alveolar ridge bone.

### DETAILED DESCRIPTION OF THE INVENTION

The present invention includes a scaffold or carrier matrix which comprises degradable polymer, preferably collagen, and ceramic materials. The carrier matrix has a scaffold structure and is incorporated with growth factors that stimulate the generation of new bone growth. The ceramic materials comprise calcium compounds. For example, calcium compounds may comprise calcium carbonate, calcium sulfate, calcium lactobionate, calcium fluorite, calcium fluorophosphates, calcium chlorophosphate, calcium chloride, calcium lactate, hydroxyapatite, ceramics, calcium oxide, calcium monophosphate, calcium diphosphate, tricalcium phosphate, calcium silicate, calcium metasilicate, calcium silicide, calcium acetate, and biphasic calcium phosphate.

Biphasic calcium phosphate is the preferred ceramic, with a desirable biphasic calcium phosphate having a tricalcium phosphate:hydroxyapatite weight ratio from about 50:50 to about 95:5. More preferable about 70:30 to about 95:5, even more preferably about 80:20 to about 90:10, and most preferably about 85:15. The ceramic material has an approximate porosity of at least 20%. Generally, the amount of mineral in the biomedical implant must be sufficient to allow for the formation of an osteoid in the bone void or target site. Further, the composition of the carrier matrix must be such that the scaffold is maintained for a sufficient amount of time for osteoid formation and eventual bone formation.

Various types of available collagen are suitable for the carrier matrix. The collagen and ceramic particles, forming a scaffold architecture, constitute a scaffold or carrier matrix. The collagen may be purchased commercially or prepared by methods known in the art. Both fibrillar and non-fibrillar collagen may be used. In addition to collagen, gelatin and other types of natural and synthetic polymers are also suitable.

When placed in a bone void, the carrier matrix with its porous structure provides scaffolding for the migration, transformation, and attachment of new bone tissue cells. During the process of osteogenesis the carrier matrix is gradually replaced with bone tissue as the injury site is repaired.

The osteogenic implant primarily stimulates osteoblasts, which are responsible for formation of new bone tissue. To facilitate the growth of new bone the preferred embodiment is a carrier matrix with a high mineral content. The high mineral content primarily ensures that enough ceramic is available as new bone formation progresses at the target site and that bone generation occurs before the scaffold is degraded away. Further, the necessary level of mineral content required in the composition will also partially depend on the level of osteogenic activity. That is, the higher the growth factor activity level the greater the mineral content required to maintain bone formation.

In preferred embodiments of the invention, the ratio of particulate mineral to resorbable carrier matrix is at least 3:1 by weight but more preferably at least 10:1. In particularly preferred embodiments, the particulate mineral will constitute at least 95% by weight of the implant. Highly effective biomedical implants comprise about 97% to 99% of particulate mineral by weight and about 1% to about 3% collagen or other polymer matrix material. Further, the mineral component with an average particle size of at least 0.1 mm is preferred, but more preferably about 0.5 mm to about 2 mm, and most preferably about 0.5 mm to about 1.5 mm.

The biomedical implant is useful in a variety of diseases, disorders, and defects where new bone formation is an essential part of the therapy. The biomedical implant is useful for long bone defects such as in the femur, tibia, fibula, humerus, etc. or also for vertebral body defects. The implant is particularly useful in periodontal diseases where the alveolar bone requires additional new bone growth to support dental implants. Essentially the implant is especially useful where overlying soft tissues covers the target area or defect. The preformed implant with pre-attached solid impermeable membrane will facilitate new bone formation without the interfering soft tissue infiltration into the biomedical implant.

The resorbable biomedical implant contains an impermeable barrier, preferably a type of membrane, integrally attached to a portion of the surface of the carrier matrix. A solid impermeable barrier membrane will resist the passage of soft tissue cells that may potentially migrate into the porous carrier matrix. Soft tissue cells, such as muscle cells, connective tissue, fibroblasts, or mast cells can infiltrate the porous carrier matrix. Further, an inflammatory response may be present at the site of injury or implant site and additional cell types and cellular components, including but not limited to neutrophils, monocytes, lymphocytes, eosinophils, basophils, and proteoglycans may infiltrate the implant post-surgically. The portion of the implant exposed to the soft tissue will have a solid impermeable membrane integrally attached to the biomedical implant to prevent the movement of cells and cellular components into the porous areas of the implant and thus facilitate osteogenesis at the intended target site.

The membrane barrier may be made of natural or synthetic resorbable and degradable polymers, such as polylactic acid (PLA), polyglycolyic (PGA), polyorthoester (POE), polyglycolide, polylactide, polylactide-polyglycolide copolymers, collagen or other resorbable polymers and co-polymers. Biogradable PLA/PGA polymers and their copolymers represent a family of materials having a wide range of differing bioengineering properties and concomitant biological responses. The preferred polymer for the instant biomedical implant is collagen.

The biomedical implant is made by preparing a collagen slurry and an appropriately sized impermeable membrane. The collagen slurry may be formed as known in the art, also described in U.S. patent application Ser. No. 09/923,116, herein incorporated by reference in its entirety.

To make the sponge implant, a collagen slurry may be formed as known in the art. The collagen slurry is preferably chilled to increase its viscosity to help suspend the porous particulate mineral component. The porous particulate mineral or ceramic particles are dispersed into the collagen slurry and gently mixed. In a preferred embodiment a solid impermeable membrane, preferable collagen, is laid in a sterile tray or other form. After the porous particulate mineral component is uniformly dispersed in the slurry, the slurry is poured over the solid membrane and then freeze dried. The freeze drying causes sublimation of the water molecules leaving behind numerous pores and also causing some cross-linking of the collagen fibrils. Cross-linking occurs among the collagen fibrils in the slurry and also with the collagen of the solid impermeable membrane. After freeze drying, preferably the implant is exposed to a vapor formaldehyde deposition as a further cross-linking agent for the collagen. The composite formed is generally three-dimensionally stable with the solid membrane an integral part of the sponge type biomedical implant. That is, the solid membrane and the carrier matrix are inextricably interconnected to form a single unit. The solid membrane occludes a portion of the pores of the biomedical implant forming a barrier to soft-tissue ingrowth. The implant can then be sterilized and packaged in accordance with known procedures.

The freeze dried scaffold matrix is re-hydrated with a biocompatible solution such as saline, ringer's solution, water or other substance compatible with the implant site of the patient. The re-hydrating solution is incorporated with an effective amount of growth factors, antibiotics, anti-inflammatory, analgesics or any other therapeutic or biocompatible agents. The subsequent pores created by freeze drying are thus filled with therapeutic agents during re-hydration. The resulting biomedical implant is then mechanically shaped and fitted to the implant site.

An essential aspect of the invention is to maintain a sufficient three dimensional architectural structure that will support the overlying soft-tissue without significant compression of the biomedical implant. Significant compression will cause distortion of the impermeable membrane and may further partially occlude some of the pores of the carrier matrix and decrease the osteogenic effectiveness of the biomedical implant. Yet the biomedical implant must still be sponge-like to facilitate the implantation process in bone void areas. The solid impermeable membrane attachment to the carrier matrix should not significantly alter the structural characteristics of the implant. A regional alteration in the structural properties of the biomedical implant due to the solid impermeable membrane attachment may affect the local compression properties of the biomedical implant and alter the osteogenic properties of the biomedical implant. This is an issue with a synthetically mineralized scaffold matrix, that is, non-bone derived mineralization as used in this invention. However, using the preferred embodiments as described herein will yield the desired consistency to achieve a structurally sound, yet an osteogenic pliable, biomedical implant.

In another aspect of the invention, the carrier matrix is formed as above without the solid impermeable membrane placed in the tray or other form type. The solid impermeable membrane, preferably collagen, is then attached to the surface portion of the sponge type carrier matrix desired for occlusion. Various methods of attaching the solid membrane to the carrier matrix may be used. For example, suitable biocompatible binding agents or glues may be applied to the carrier matrix, impermeable membrane surface or both, such as biological adhesives, cyanoacrylates, epoxy based substances, dental resin cements, and various resorbable and non-resorbable polymers. A preferred glue is collagen or a collagen based substance. The implant can then be air dried, and more preferably freeze dried.

In another embodiment a plasticizer may be used to obtain a desired consistency of the spongy carrier matrix. Plasticizers include, but not limited to, polyhydroxy compounds such as a carbohydrate, a polyhydroxy aldehyde, a polyhydroxy ketone, a glycogen, an aldose, a sugar, a mono- or polysaccharide, an oligosaccharide, a polyhydroxy carboxylic compound, polyhydroxy ester compound, a cyclodextrin, a polyethylene glycol polymer, a glycerol an alginate, a chitosan, a polypropylene glycol polymer, a polyoxyethylene-polyoxypropylene block co-polymer, agar, and hyaluronic acid or polyhydroxy derivative compounds.

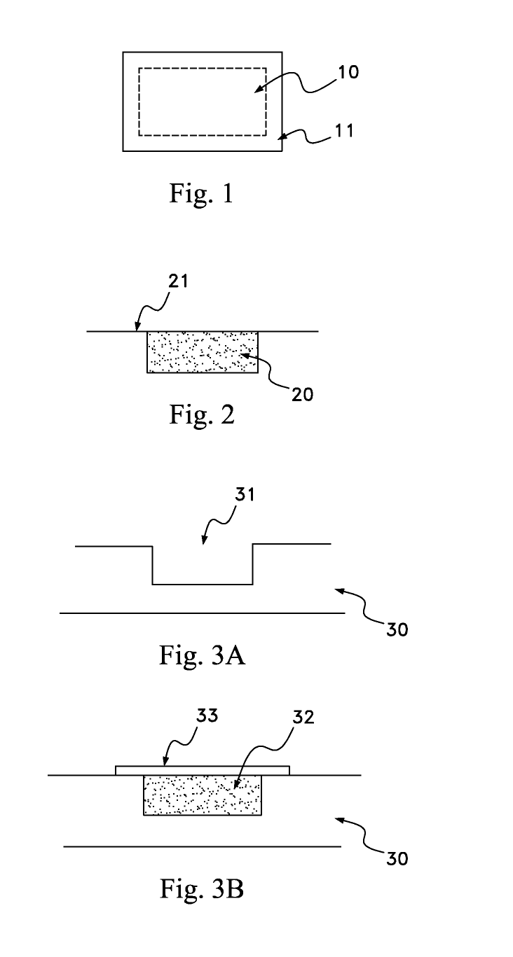

The dimensions of the implant produced may vary depending on the application. Dimensions of a typical sponge are, for example, about 4 cm (length) by 2 cm (width) by 1 cm. However, the sponge is preferably mechanically shaped by the surgeon to fit any bone void configuration. FIG. 1 shows a top view of the implant with the solid impermeable membrane 11 overlapping the sponge or bone void filler part 10 of the implant. A side or cross-sectional view shows the porous bone void filler 20 and the solid impermeable membrane 21 that will prevent the porous osteogenic implant surface from directly contacting the surrounding soft tissues.

After the implant is freeze dried, cross-linked, and sterilized it may be incorporated with osteogenic factors. Preferred compositions of the invention may include an osteoinductive factor, such as an osteoinductive protein or a nucleotide sequence encoding an osteoinductive protein operably associated with a promoter (e.g. provided in a vector such as a viral vector) which drives expression of the gene in the animal recipient to produce an effective amount of the protein. As discussed above, the osteogenic factor utilized in the present invention can be one that stimulates production or activity of the osteoblasts. The factor is preferably a bone morphogenetic protein (BMP) or a LIM mineralization protein (LMP), or comprises a nucleotide sequence encoding a BMP or LMP or any combination thereof. Recombinant human BMPs are preferred, and may be commercially obtained or prepared as described and known in the art, e.g. in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,932 to Wang et al.; U.S. Pat. No. 5,116,738 to Wang et al.; U.S. Pat. No. 5,013,649 to Wang et al.; U.S. Pat. No. 5,106,748 to Wozney et al; and PCT Patent Nos. WO93/00432 to Wozney et al.; WO94/2693 to Celeste et al.; and WO94/26892 to Celeste et al. Further, the osteoinductive factor may be isolated from bone. Methods for isolating BMP from bone are described in U.S. Pat. No. 4,294,753 to Urist and Urist et al., PNAS 371, 1984. Recombinant human BMP-2 (rhBMP-2), recombinant human BMP-4 (rhBMP-4), BMP-6, rhBMP-6, BMP-7[OP-1] recombinant human BMP-7 (rhBMP-7), Nell-1, recombinant human growth differentiation factor (rhGDF-5), statins, or heterodimers thereof are more preferred. However, the most preferred growth factors are rhBMP-2, rhBMP-7, and rhGDF-5. The osteoinductive factor may also be LMP or a suitable vector incorporating a gene encoding the same operably associated with a promotor, as described in WO99/06563 (see also genbank accession No. AF095585). When such vectors are employed as osteogenic factors in accordance with the invention, they are preferably delivered in conjunction with cells, for example autologous cells from the recipient of the implant. Most preferably the vector is delivered in conjunction with autologous white blood cells derived from bone marrow or peripheral blood of the recipient. These cells may be applied to the sponge composition along with the osteogenic factor prior to implantation.

Further, as an example, BMP or other osteogenic factors may be included in the formed sponge by combining the BMP with a liquid carrier as known in the art and infusing the liquid into the sponge.

In further enhancements of the compositions of the present invention, other growth factors or osteogenic enhancing factors may be incorporated into the composition. Such additional factors include host compatible osteogenic progenitor cells, autographic bone marrow, allographic bone marrow, transforming growth factor-beta (TGF-β), fibroblast growth factor (FGF), platelet-derived growth factor (PDGF), insulin-related growth factor (IGF-I), insulin-related growth factor-II (IGF-II) beta-2-microglobulin (BDGF II), PTH, PGE2 agonist, granulocyte-colony stimulating factor (G-CSF), vascular endothelial growth factor (VEGF), mesenchymal stem cells (MSC), matrix metalloproteinase (MMP), peptides, a statin, antibiotics and steroids.

Additional enhancements may comprise an effective amount of anti-inflammatory agents, such as anti-cytokine agents. Anti-cytokine agents may comprise TNF-a inhibitors, IL-1 inhibitors, IL-6 inhibitors, IL-8 inhibitors, IL-12 inhibitors, IL-15 inhibitors, IL-10, NF Kappa B inhibitors, and interferon-gamma (IFN-gamma).

Still further enhancements may include effective amounts of antibiotics and analgesics. These agents are well known in the art. In different embodiments of the invention, other active ingredients may also be added to the carrier matrix. An active ingredient may include an antimicrobial, antifungal, antiviral, an antineoplastic agent, an antibiotic, an analgesic, narcotic antagonists, and any combination thereof, in addition to one or more anti-cytokine agents.

A suitable agent may include an analgesic such as morphine, a suitable narcotic antagonist (e.g., naloxone), local anaesthetics (e.g., lidocaine, bupivacaine, mepivacaine, dibucaine, prilocaine, etidocaine, ropivacaine, procaine, tetracaine, etc.), glutamate receptor antagonists, adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the contents of which are incorporated herein by reference.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol and any combination thereof.

In other embodiments, the anti-cytokine agents, and optionally any other agent, may be presented in a sustained-release formulation. Such sustained release formulations are well known in the art.

The biomedical implant is particularly suitable for alveolar bone defects, periodontal surgeries, oral maxillofacial procedures, plastic and reconstructive surgery, guided bone regeneration, modifying bone contours, filling of cranial facial defects, long bone defects (e.g., femur, tibia, humerus, etc.) or other skeletal applications. In guided tissue regeneration, e.g., in oral maxillofacial surgeries, the biomedical implant of the present invention does not require a separate solid membrane barrier overlaid and sutured in place to prevent soft tissue infiltration. The biomedical implant with the integral occlusive solid membrane barrier does not require a separate means of attachment. FIG. 3A shows a diagrammatic cross-sectional or side view of alveolar bone 30 and a corresponding defect 31. FIG. 3B shows the alveolar bone defect filled with a sponge type biomedical implant 32 and the solid membrane barrier 33 attached to the implant and overlying a portion of the alveolar bone beyond the defect, thus guiding tissue regeneration and preventing the ingrowth of soft tissue components into the porous implant 32.

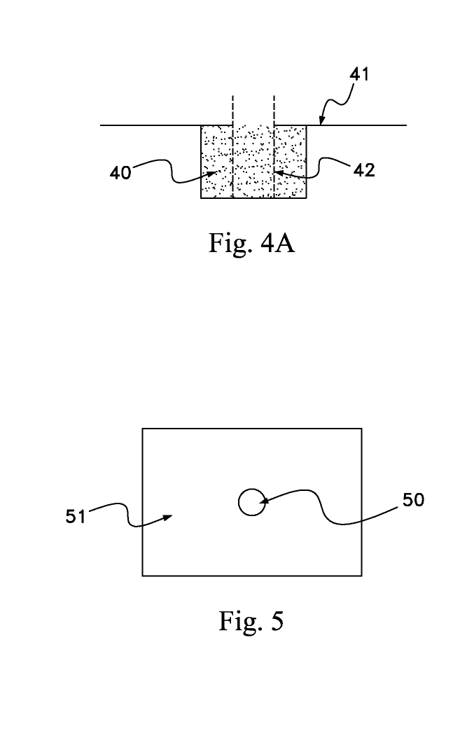

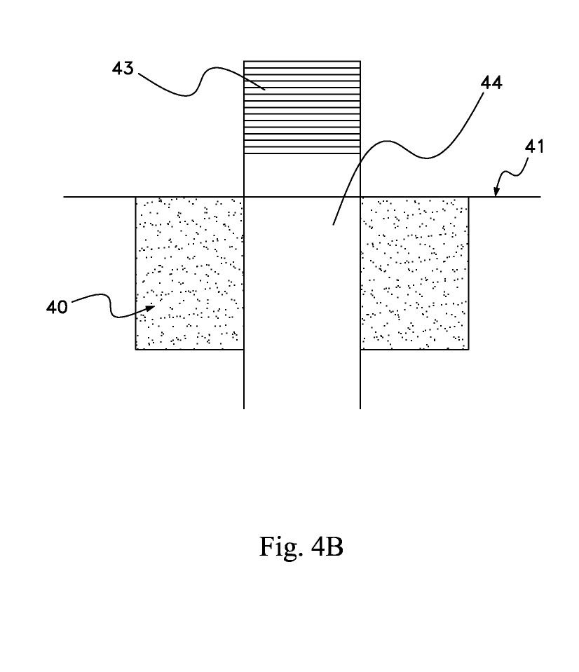

FIG. 5 shows a top view of an alternative design for a periodontal application. The biomedical implant has a cut-out or hole 50 placed in the implant to allow a tooth structure to emerge through the implant and permit the osteogenic carrier matrix to contact the intended target site or fill in the bone void areas below the tooth structure. In this configuration, the top view is the top surface of the solid membrane 51 integrally attached to the sponge carrier matrix. FIG. 4A is a diagrammatic cross-sectional or side view of the periodontal implant, with the sponge-type carrier matrix 40, the tooth structure 42 piercing through the carrier matrix 40 and solid membrane 41. FIG. 4B illustrates the use of a dental implant 44 with a threaded portion 43 for attachment of the prosthesis or tooth. The biomedical implant 40 restores the alveolar bone and the dental implant 44 is inserted through the regenerated alveolar bone.

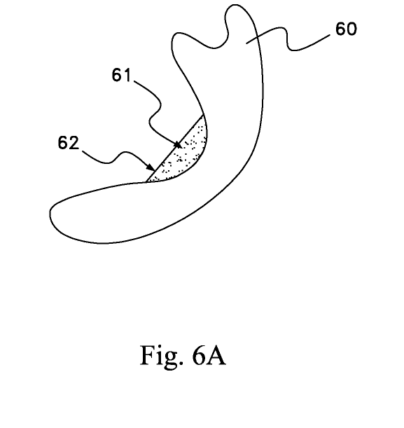

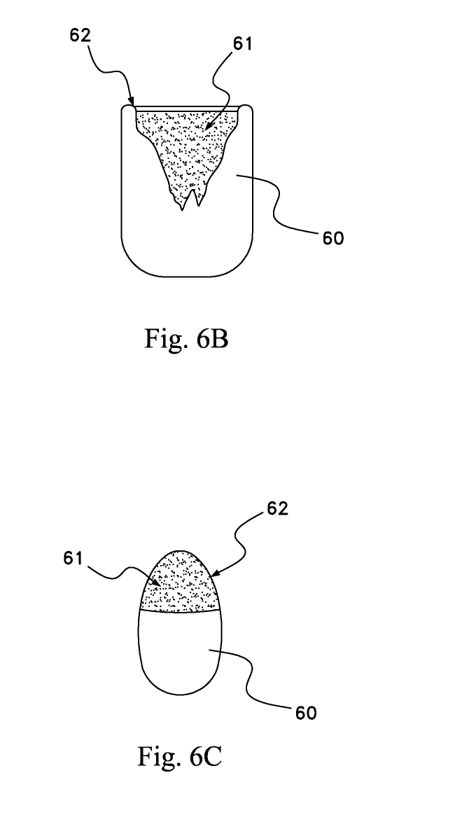

FIG. 6A shows a diagrammatic side view and yet another application for alveolar bone. For curvilinear alveolar bone 60 that requires a build up of new bone the biomedical implant 61 is positioned for new bone growth. The solid impermeable membrane 62 separates the overlying soft tissue from the implant. The biomedical implant 61 causes new bone tissue growth and restores the bone tissue level or provides sufficient new bone for accommodating dental implants. FIG. 6A may consist of an alveolar bone defect, an augmentation of the alveolar ridge bone or both. FIG. 6B is a cross-sectional view through the center of the biomedical implant 61 of FIG. 6A with an alveolar bone defect. The biomedical implant 61 in FIG. 6B is shown filling the defect of the alveolar bone 60 with the solid impermeable membrane 62 providing separation from the overlying soft tissues. FIG. 6C is a cross-sectional view through the center of the biomedical implant 61 of FIG. 6A where an augmentation of the alveolar ridge bone is required. FIG. 6C shows the alveolar bone 60 with the biomedical implant 61 positioned to restore the alveolar ridge, and the solid impermeable membrane 62 following the contoured shape of the alveolar bone to effectively separate the overlying soft tissues from the biomedical implant 61.

The solid impermeable membrane often may extend beyond the biomedical implant as shown in FIGS. 1,2,3B,4A,4B. As an additional embodiment, a fibrin type glued (fibrinogen derived) may be used to obtain a sufficient seal between the solid impermeable membrane and the surrounding bone or biological structure. This may prevent any potential fluids or tissue components from infiltrating beneath the solid impermeable membrane where it does not contact biomedical implant.

U.S. patent application Ser. No. 09/923,116 (the '116 application) herein incorporated by reference in its entirety describes additional compositions and uses of a resorbable osteogenic implant for bone grafting.

The kit form of the invention comprises the freeze dried scaffold matrix with the solid impermeable membrane attached, with growth factors, antibiotics, analgesics, anti-inflammatory agents or other therapeutic biocompatible agents. The freeze dried scaffold is re-hydrated with a biocompatible solution, such as saline, ringer's solution, or other blood-tissue compatible substance or combination thereof. The growth factors or other biocompatible agents are incorporated with the re-hydrating solution so as to fill the extensive number of pores previously create during freeze drying of the scaffold matrix.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed:

1. A biomedical implant comprising:
a synthetically mineralized scaffold matrix;
a solid membrane integrally bonded to the scaffold matrix; and
an effective amount of an osteogenic factor incorporated into the scaffold matrix to cause new bone growth.

2. The biomedical implant according to claim 1, wherein the synthetically mineralized scaffold matrix comprises an effective amount of at least one particulate mineral having an average particle diameter of at least 0.1 mm.

3. The biomedical implant according to claim 1, wherein the scaffold matrix comprises collagen.

4. The biomedical implant according to claim 1, wherein the scaffold collagen matrix comprises soluble and insoluble collagen.

5. The biomedical implant according to claim 2, wherein the particulate mineral forms ceramic particles.

6. The biomedical implant according to claim 5, wherein the ceramic particles comprise biphasic calcium phosphate or hydroxyapatite and beta-tri-calcium phosphate formulations.

7. The biomedical implant according to claim 5, wherein the ceramic has a porosity of at least about 20%.

8. The biomedical implant according to claim 5, wherein the particulate mineral has an average particle size from about 0.1 mm to about 2.0 mm.

9. The biomedical implant according to claim 5, wherein the particulate mineral has an average particle size from about 0.5 mm to about 1.5 mm.

10. The biomedical implant according to claim 2, wherein the particulate mineral is present in about a 3:1 weight ratio with respect to the scaffold matrix.

11. The biomedical implant according to claim 2, wherein the particulate mineral comprises at least about 95% by weight of the biomedical implant.

12. The biomedical implant according to claim 2, wherein the particulate mineral comprises calcium compounds.

13. The biomedical implant according to claim 12, wherein the calcium compounds comprise at least one of: calcium sulfate, calcium carbonate, calcium fluorite, calcium fluorophosphates, calcium chlorophosphate, calcium chloride, calcium lactate, hydroxyapatite, ceramics, calcium oxide, calcium monophosphate, calcium diphosphate, tricalcium phosphate, calcium silicate, calcium metasilicate, calcium acetate, and biphasic calcium phosphate or any combination thereof.

14. The biomedical implant according to claim 1, further comprising an effective amount of a polyhydroxy compound.

15. The biomedical implant according to claim 14, wherein the polyhydroxy compound comprises at least one of: a carbohydrate, a polyhydroxy aldehyde, a polyhydroxy ketone, a glycogen, an aldose, a sugar, a mono- or polysaccharide, an oligosaccharide, a polyhydroxy carboxylic compound, polyhydroxy ester compound, a cyclodextrin, a polyethylene glycol polymer, glycerol, an alginate, a chitosan, a polypropylene glycol polymer, a polyoxyethylene-polyoxypropylene block co-polymer, agar, and hyaluronic acid or polyhydroxy derivative compounds.

16. The biomedical implant according to claim 15, wherein the polyhydroxy derivative compounds include mono and diesters of glycerol.

17. The biomedical implant according to claim 1, wherein the solid membrane comprises a biodegradable polymer comprising collagen, polylactic acid (PLA), polyglycolic (PGA), or polyorthoester (POE).

18. The biomedical implant according to claim 1, wherein the scaffold matrix further comprises an effective amount of a growth factor, an anti-inflammatory agent, an antibiotic, an analgesic, or any combination thereof.

19. The biomedical implant according to claim 18, wherein an effective amount of a growth factor comprises at least one of: BMP-2, rhBMP-2, BMP-4, rhBMP-4, BMP-6, rhBMP-6, BMP-7[OP-1], rhBMP-7, GDF-5, rhGDF-5, Nell-1, LIM mineralization protein, platelet derived growth factor (PDGF), transforming growth factor β (TGF-β), insulin-related growth factor-I (IGF-I), insulin-related growth factor-II (IGF-II), fibroblast growth factor (FGF), beta-2-microglobulin (BDGF II), PTH, PGE2 agonist, granulocyte-colony stimulating factor (G-CSF), vascular endothelial growth factor or (VEGF), mesenchymal stem cell(MSC) matrix metalloproteinase (MMP), or a statin.

20. The biomedical implant according to claim 18, wherein an effective amount of an anti-inflammatory agent comprises anti-cytokine agents.

21. The biomedical implant according to claim 20, wherein the anti-cytokine agents comprise at least one of: TNF-a inhibitors, IL-1 inhibitors, IL-6 inhibitors, IL-8 inhibitors, IL-12 inhibitors, IL-15 inhibitors, IL-10, NF Kappa B inhibitors, and Interferon-gamma (IFN-gamma).

22. A method of forming the biomedical implant comprising:
providing a synthetically mineralized scaffolding matrix;
providing a solid membrane substantially impermeable to soft tissue;
attaching the solid impermeable membrane to at least a portion of the surface of the synthetically mineralized scaffolding matrix.

23. The method of forming the biomedical implant according to claim 22, wherein providing the synthetically mineralized scaffold matrix comprises:
pouring a collagen-ceramic slurry into a tray or mold, the collagen-ceramic slurry having a particulate mineral size of an average particle diameter of at least about 0.1 mm, and having at least about a 3:1 weight ratio relative to the scaffold matrix; and
freeze drying the collagen-ceramic slurry.

24. The method of forming the biomedical implant according to claim 22, wherein attaching the scaffold matrix to the solid impermeable membrane comprises:
placing the solid impermeable membrane in the tray or mold;
pouring the collagen-ceramic slurry over the solid impermeable membrane; and
freeze drying the collagen-ceramic slurry with the sold impermeable membrane.

25. The method of forming the biomedical implant according to claim 22, wherein attaching the scaffold matrix to the solid impermeable membrane comprises:
applying biocompatible binding agents to the solid impermeable membrane or to a portion of the surface of the scaffold matrix or both; and
attaching the solid impermeable membrane to the scaffold matrix for binding to occur.

26. The method according to claim 22, wherein the solid membrane comprises a biodegradable polymer comprising collagen, polylactic acid (PLA), polyglycolic (PGA), or polyorthoester (POE).

27. The method according to claim 23, wherein the collagen-ceramic slurry comprises collagen, biphasic calcium phosphate or hydroxyapatite and beta-tri-calcium phosphate formulations.

28. A biomedical implant kit comprising:
a preformed scaffold matrix with a solid impermeable membrane integrally attached;
an effective amount of at least one of: growth factors, antibiotics, analgesics, anti-inflammatory agents or any combination thereof; and
a re-hydrating solution to incorporate the growth factors antibiotics, analgesics, or anti-inflammatory agents into the scaffold matrix.
